(19) United States
(12) Patent Application Publication
(10) Pub. No.: US 2026/0034294 A1
(43) Pub. Date: Feb. 5, 2026

(54) PROTECTION AND ISOLATION OF SENSING CIRCUITRY FOR NEGATIVE PRESSURE WOUND THERAPY DEVICES

(71) Applicant: T.J.Smith and Nephew,Limited, Hull (GB)

(72) Inventors: Allan Kenneth Frazer Grugeon Hunt, Beverley (GB); Felix Clarence Quintanar, Hull (GB)

(21) Appl. No.: 19/099,700

(22) PCT Filed: Sep. 11, 2023
(86) PCT No.: PCT/EP2023/074925

§ 371 Date: Jan. 29, 2025

(30) Foreign Application Priority Data
Sep. 27, 2022 (GB) 2214074.3

(51) Int. Cl. A61M 1/00

(52) U.S. Cl. CPC A61M 1/982; A61M 2202/0014; A61M 2202/04; A61M 2205/3327; A61M 2205/3344; A61M 2205/3379; A61M 2205/3592; A61M 2205/50

(57) ABSTRACT

A negative pressure wound therapy system can include a fluid level detection circuitry positioned in a canister. The fluid level detection circuitry can include a pair of electrodes and a logic circuitry configured to detect fluid level based on one or more signals from the pair of electrodes. The logic circuitry can be protected and isolated from the patient's body to prevent flow of any leakage current through the patient's body using one or more approaches disclosed herein.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy system can include a negative pressure wound therapy device with a negative pressure source configured to aspirate, via a fluid flow path, fluid from a wound covered by a wound dressing. The system can include a canister positioned in the fluid flow path and configured to store at least some of the fluid aspirated by the negative pressure source. The system can include a fluid level sensing circuitry positioned at least partially in the canister. The fluid level sensing circuitry can include first and second electrodes configured to come into contact with fluid stored in the canister when the fluid reaches the first and second electrodes. The fluid level sensing circuitry can include an integrated circuit (IC) with first and second connectors connected to the first and second electrodes. The IC can be configured to determine a level of fluid stored in the canister based on a signal received from the first and second electrodes. The fluid level sensing circuitry can include first and second resistors connected to the first and second connectors and configured to protect against leakage current from the fluid level sensing circuitry.

The negative pressure wound therapy system of any of the preceding paragraphs and/or any of the apparatuses, systems, or devices disclosed herein can include one or more of the following features. Impedance across the first and second electrodes can decrease responsive to the fluid in the canister reaching the first and second electrodes. The IC can be configured to determine the level of fluid responsive to detecting that the impedance across the first and second electrodes is below an impedance threshold indicative of a short circuit across the first and second electrodes. The short circuit can be formed by the fluid in the canister reaching the first and second electrodes. Impedance of the first and second resistors can be lower than the impedance threshold. The impedance of the first and second resistors can be 15-30% of the impedance threshold.

The negative pressure wound therapy system of any of the preceding paragraphs and/or any of the apparatuses, systems, or devices disclosed herein can include one or more of the following features. The first and second connectors can include first and second pins of the IC. The fluid level sensing circuitry can include an antenna configured to wirelessly communicate the level of fluid stored in the canister to the negative pressure wound therapy device. The IC can include third and fourth connectors connected to the antenna. The fluid sensing circuitry can include third and fourth resistors connected to the third and fourth connectors and configured to protect against leakage current. The third and fourth connectors can include third and fourth pins of the IC.

The negative pressure wound therapy system of any of the preceding paragraphs and/or any of the apparatuses, systems, or devices disclosed herein can include one or more of the following features. The fluid level sensing circuitry can include at least one first diode connected to the first and second connectors and configured to protect against electrostatic discharge (ESD). The at least one first diode can include a first bidirectional transient voltage suppressor (TVS) diode connected across the first and second connectors. The fluid level sensing circuitry can include at least one second diode connected to the third and fourth connectors and configured to protect against ESD. The at least one second diode can include a second bidirectional TVS diode connected across the third and fourth connectors.

The negative pressure wound therapy system of any of the preceding paragraphs and/or any of the apparatuses, systems, or devices disclosed herein can include one or more of the following features. The IC can be coated or encapsulated in a non-conductive material. The negative pressure wound therapy device can include electronic circuitry configured to monitor orientation of at least one of the negative pressure wound therapy device or the canister. The fluid level sensing circuitry can be configured to receive power wirelessly from the electronic circuitry of the negative pressure wound therapy device. The electronic circuitry of the negative pressure wound therapy device can be configured to not provide power to the fluid level sensing circuitry responsive to a detection that at least one of the negative pressure wound therapy device or the canister is in an incorrect orientation for aspirating fluid from the wound. Incorrect orientation can include orientation other than vertical orientation. The fluid level sensing circuitry can be configured to operate as a passive near field communication (NFC) device. The level of fluid stored in the canister can indicate that the canister is full or nearly full.

The negative pressure wound therapy system of any of the preceding paragraphs and/or any of the apparatuses, systems, or devices disclosed herein can include one or more of the following features. The canister can include a cap configured to be attached to a housing of the negative pressure wound therapy device. The fluid level sensing circuitry can be positioned in the cap. The first and second electrodes can be oriented downward toward an interior volume of the canister. The fluid level sensing circuitry can be positioned between the first and second electrodes. The IC can be reinforced with a base material. The base material can include fiberglass with epoxy (FR4).

A negative pressure wound therapy system can include a negative pressure wound therapy device with a negative pressure source configured to aspirate, via a fluid flow path, fluid from a wound of a patient covered by a wound dressing. The system can include a canister positioned in the fluid flow path and configured to store at least some of the fluid aspirated by the negative pressure source. The system can include a fluid level sensing circuitry positioned at least partially in the canister. The fluid level sensing circuitry can include first and second electrodes configured to come into contact with fluid stored in the canister when the fluid reaches the first and second electrodes. The fluid level sensing circuitry can include a processing circuitry with first and second connectors connected to the first and second electrodes. The processing circuitry can be configured to be powered on for a duration of time that is sufficient to determine the level of fluid stored in the canister and does not exceed a threshold duration of time associated with a minimum duration of time that the patient can be safely exposed to a leakage current. The fluid level sensing circuitry can include first and second resistors connected to the first and second connectors and configured to protect the patient against leakage current.

The negative pressure wound therapy system of any of the preceding paragraphs and/or any of the apparatuses, systems, or devices disclosed herein can include one or more of the following features. Impedance across the first and second electrodes can decrease responsive to the fluid in the canister reaching the first and second electrodes. The processing circuitry can be configured to determine the level of fluid responsive to detecting that the impedance across the first and second electrodes is below an impedance threshold indicative of a short circuit across the first and second electrodes. The short circuit can be formed by the fluid in the canister reaching the first and second electrodes. Impedance of the first and second resistors can be lower than the impedance threshold. The impedance of the first and second resistors can be 15-30% of the impedance threshold.

The negative pressure wound therapy system of any of the preceding paragraphs and/or any of the apparatuses, systems, or devices disclosed herein can include one or more of the following features. The processing circuitry can include an integrated circuit (IC), and the first and second connectors can include first and second pins of the IC. The fluid level sensing circuitry can include an antenna configured to wirelessly receive power from the negative pressure wound therapy device and communicate the level of fluid stored in the canister to the negative pressure wound therapy device. The processing circuitry can include third and fourth connectors connected to the antenna. The fluid sensing circuitry can include third and fourth resistors connected to the third and fourth connectors and configured to protect against leakage current. The processing circuitry can include an integrated circuit (IC), and the third and fourth connectors can include third and fourth pins of the IC.

The negative pressure wound therapy system of any of the preceding paragraphs and/or any of the apparatuses, systems, or devices disclosed herein can include one or more of the following features. The fluid level sensing circuitry can include at least one first diode connected to the first and second connectors and configured to protect the fluid level sensing circuitry against electrostatic discharge (ESD). The at least one first diode can include a first bidirectional transient voltage suppressor (TVS) diode connected across the first and second connectors. The fluid level sensing circuitry can include at least one second diode connected to the third and fourth connectors and configured to protect the fluid level sensing circuitry against ESD. The at least one second diode can include a second bidirectional TVS diode connected across the third and fourth connectors.

The negative pressure wound therapy system of any of the preceding paragraphs and/or any of the apparatuses, systems, or devices disclosed herein can include one or more of the following features. The processing circuitry can be coated or encapsulated in a non-conductive material. The negative pressure wound therapy device can include electronic circuitry configured to monitor orientation of at least one of the negative pressure wound therapy device or the canister. The fluid level sensing circuitry can be configured to receive power wirelessly from the electronic circuitry of the negative pressure wound therapy device. The electronic circuitry of the negative pressure wound therapy device can be configured to not provide power to the fluid level sensing circuitry responsive to a detection that at least one of the negative pressure wound therapy device or the canister is in an incorrect orientation for aspirating fluid from the wound. Incorrect orientation can include orientation other than vertical orientation. The fluid level sensing circuitry can be configured to operate as a passive near field communication (NFC) device. The level of fluid stored in the canister can indicate that the canister is full or has reached a threshold fluid level.

The negative pressure wound therapy system of any of the preceding paragraphs and/or any of the apparatuses, systems, or devices disclosed herein can include one or more of the following features. The canister can include a cap configured to be attached to a housing of the negative pressure wound therapy device. The fluid level sensing circuitry can be positioned in the cap. The first and second electrodes can be oriented downward toward an interior volume of the canister. The fluid level sensing circuitry can be positioned between the first and second electrodes. The processing circuitry can include an integrated circuit (IC) reinforced with a base material. The base material can include fiberglass with epoxy (FR4). The processing circuitry can be configured to be powered on periodically for the duration of time.

A negative pressure wound therapy system can include a negative pressure wound therapy device with a negative pressure source configured to aspirate, via a fluid flow path, fluid from a wound of a patient covered by a wound dressing. The system can include a canister positioned in the fluid flow path and configured to store at least some of the fluid aspirated by the negative pressure source. The system can include a fluid level sensing circuitry positioned at least partially in the canister. The fluid level sensing circuitry can include first and second electrodes configured to come into contact with fluid stored in the canister when the fluid reaches the first and second electrodes. The fluid level sensing circuitry can include a processing circuitry configured determine a level of fluid stored in the canister based on a signal received from the first and second electrodes. The processing circuitry can be configured to be powered on for a duration of time that is sufficient to determine the level of fluid stored in the canister and does not exceed a threshold duration of time associated with a minimum duration of time that the patient can be safely exposed to a leakage current.

The negative pressure wound therapy system of any of the preceding paragraphs and/or any of the apparatuses, systems, or devices disclosed herein can include one or more of the following features. The negative pressure wound therapy device can be configured to power on the processing circuitry and receive the level of fluid stored in the canister. The fluid level sensing circuitry can include a resistor configured to dissipate power stored by the processing circuitry.

A negative pressure wound therapy system can include a negative pressure wound therapy device with a negative pressure source configured to aspirate, via a fluid flow path, fluid from a wound of a patient covered by a wound dressing. The system can include a canister positioned in the fluid flow path and configured to store at least some of the fluid aspirated by the negative pressure source. The canister the can include an inlet configured to be connected to the negative pressure wound therapy device. The system can include a fluid level sensing circuitry positioned at least partially in the canister. The fluid level sensing circuitry can include first and second electrodes configured to come into contact with fluid stored in the canister when the fluid reaches the first and second electrodes. The fluid level sensing circuitry can include a processing circuitry configured determine a level of fluid stored in the canister based on a signal received from the first and second electrodes. The system can include an aperture structure positioned in the inlet and configured to separate a stream of fluid aspirated by the negative pressure source into drops to attain an air gap separation between the stream of the fluid and the fluid stored in the canister.

The negative pressure wound therapy system of any of the preceding paragraphs and/or any of the apparatuses, systems, or devices disclosed herein can include one or more of the following features. The aperture structure can include at least one of a curved edge, a curved edge with one or more ridges, a surface with one or more ridges, a surface one or more openings, a plurality of arms, a rotating wheel, or at least one pivoted bucket.

Disclosed herein are methods of operating a negative pressure wound therapy device of any of the preceding paragraphs and/or any of the devices, apparatuses, or systems disclosed herein.

Disclosed herein are kits that include the negative pressure wound therapy device of any of the preceding paragraphs and/or any of the devices, apparatuses, or systems disclosed herein and one or more wound dressings.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a user interface.

FIG. 3 illustrates a schematic of a control system of a negative pressure wound therapy device.

FIG. 4 illustrates another negative pressure wound therapy system.

FIG. 5 illustrates an exploded view of a canister top and associated components.

FIGS. 6A and 6B illustrate fluid level sensor electronics.

FIGS. 7A to 7C illustrate protection and isolation of fluid level sensor electronics.

FIGS. 8A and 8B illustrate mechanical separation for protection and isolation of fluid level sensor electronics.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760-X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than −X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

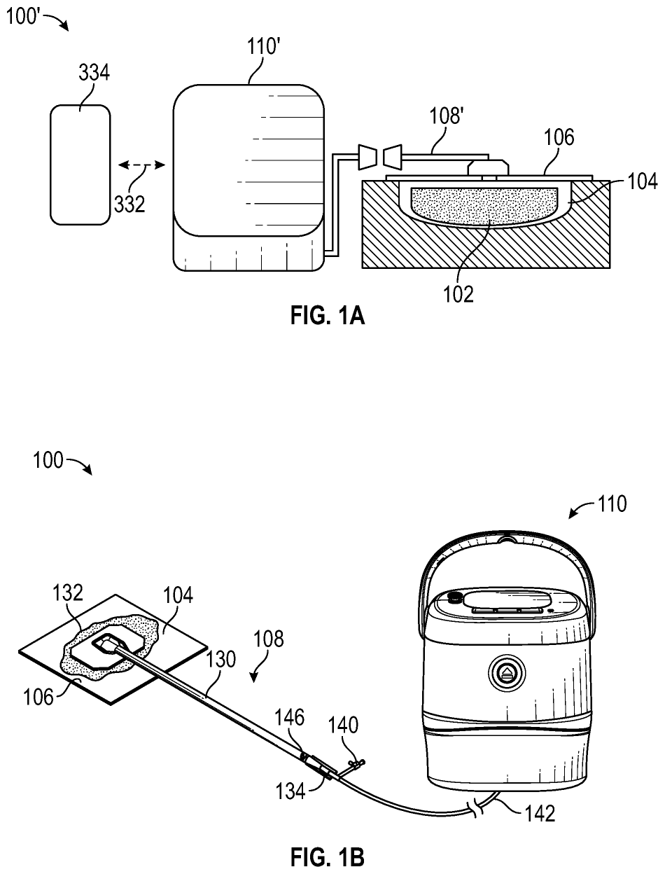

FIG. 1A schematically illustrates a negative pressure wound treatment system 100′ (sometimes referred to as a reduced or negative pressure wound therapy system, a TNP system, or a wound treatment system). In any implementations disclosed herein, though not required, the negative pressure wound treatment system 100′ can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108′ (also referred to herein as a flexible suction adapter or a fluidic connector) can be used to connect the wound cover 106 with a wound therapy device 110′ (sometimes as a whole or partially referred to as a “pump assembly”) configured to supply reduced or negative pressure. The conduit 108′ can be a single or multi lumen tube. A connector can be used to removably and selectively couple a conduit or tube of the device 110′ with the conduit 108′.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108 or any other conduit disclosed herein can be formed from polyurethane, PVC, nylon, polyethylene, silicone, or any other suitable material.

The wound cover 106 can have a port (not shown) configured to receive an end of the conduit 108. In some cases, the conduit 108 can otherwise pass through or under the wound cover 106 to supply reduced pressure to the wound 104 so as to maintain a desired level of reduced pressure in the wound 104. The conduit 108 can be any suitable article configured to provide at least a substantially sealed fluid flow pathway or path between the wound therapy device 110′ and the wound cover 106, so as to supply the reduced pressure provided by the wound therapy device 110′ to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108, to a source of negative pressure of the wound therapy device 110′. In some cases, though not required, the wound therapy device 110′ can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110′ can operate with or without the use of an exudate canister. In some cases, as is illustrated, the wound therapy device 110′ can include an exudate canister. In some cases, configuring the wound therapy device 110′ and conduit 108′ so that the conduit 108′ can be quickly and easily removed from the wound therapy device 110′ can facilitate or improve the process of wound dressing or pump changes, if necessary. Any of the pump assemblies disclosed herein can have any suitable connection between the conduit 108′ and the pump.

The wound therapy device 110′ can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110′.

As will be described in greater detail below, the negative pressure wound treatment system 100′ can be configured to provide a connection 332 to a separate or remote computing device 334. The connection 332 can be wired or wireless (such as, Bluetooth, Bluetooth low energy (BLE), Near-Field Communication (NFC), WiFi, or cellular). The remote computing device 334 can be a smartphone, a tablet, a laptop or another standalone computer, a server (such as, a cloud server), another pump device, or the like.

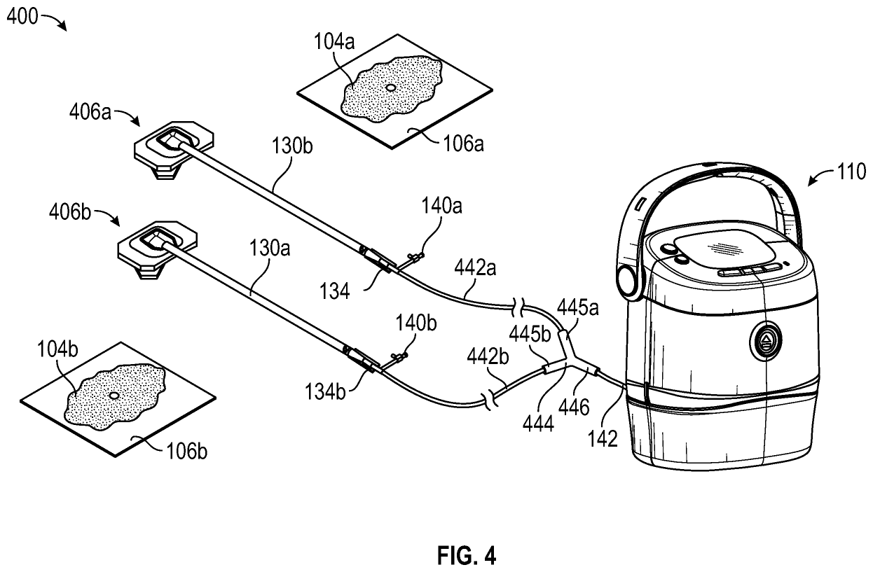

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100′ illustrated in FIG. 1A or the negative pressure wound treatment system 400 illustrated in FIG. 4, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100 can include a source of negative pressure, such as the device 110, capable of supplying negative pressure to the wound 104 through the conduit 108. Though not required, the device 110 can also include a canister or other container for the storage of wound exudates and other fluids that can be removed from the wound.

The device 110 can be connected to the connector 134 via a conduit or tube 142. In use, the applicator 132 can be placed over an aperture formed in a cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970®, or Gehring 879®) or a nonwoven material, or terry-woven or loop-pile materials. The fibers may not necessarily be woven, and can include felted and flocked (including materials such as Flotex.RTM.) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic.

In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110′ can provide negative pressure to the wound cover 106, which can be transmitted to the wound 104 via the wound filler 102. Fluid (such as, wound exudate) can be drawn through the conduit 108′ and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International App. No. PCT/EP2020/078376, each of which is incorporated by reference in its entirety as if fully set forth herein. In some cases, other suitable wound dressings can be utilized.

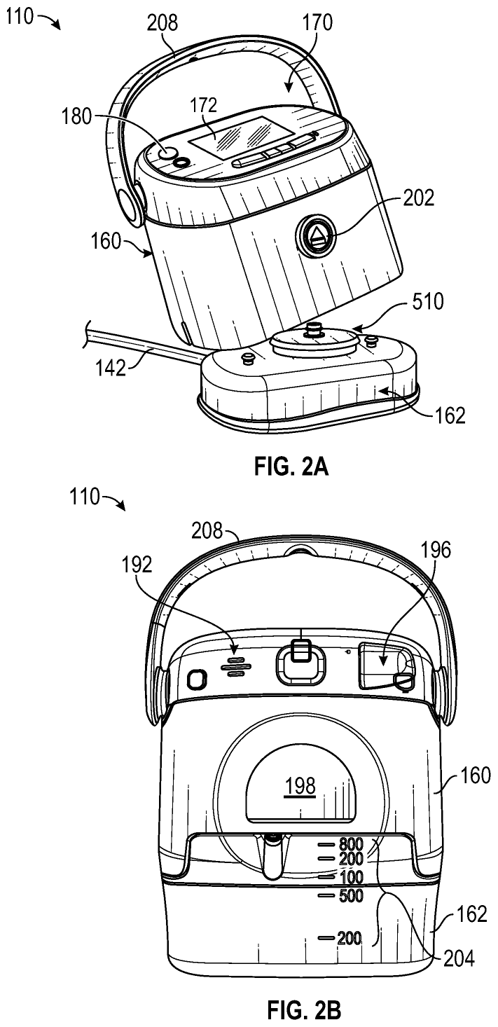

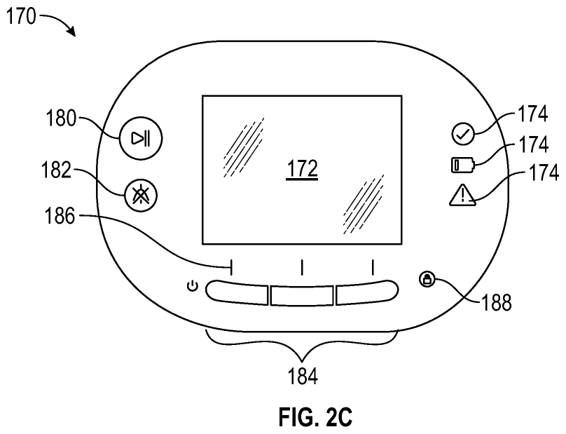

FIGS. 2A-2C show the negative pressure wound therapy device 110. As illustrated, a pump assembly 160 and canister 162 can be connected, thereby forming the wound therapy device 110. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation, low battery, a leak, canister full, blockage, overpressure, or the like). Any one or more suitable indicators can be additionally or alternatively used, such as visual, audio, tactile indicator, and so on.

FIG. 2B shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108 in order to form a fluid flow path. The canister 162 can be replaced with another canister, such as when the canister 162 has been filled with fluid. With reference to FIG. 2A, the wound therapy device 110 can include a canister inlet tube 142 (also referred to herein as a dressing port connector) in fluid communication with the canister 162. For example and without limitation, the canister inlet tube 142 can be used to connect with the conduit 108.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162.

The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

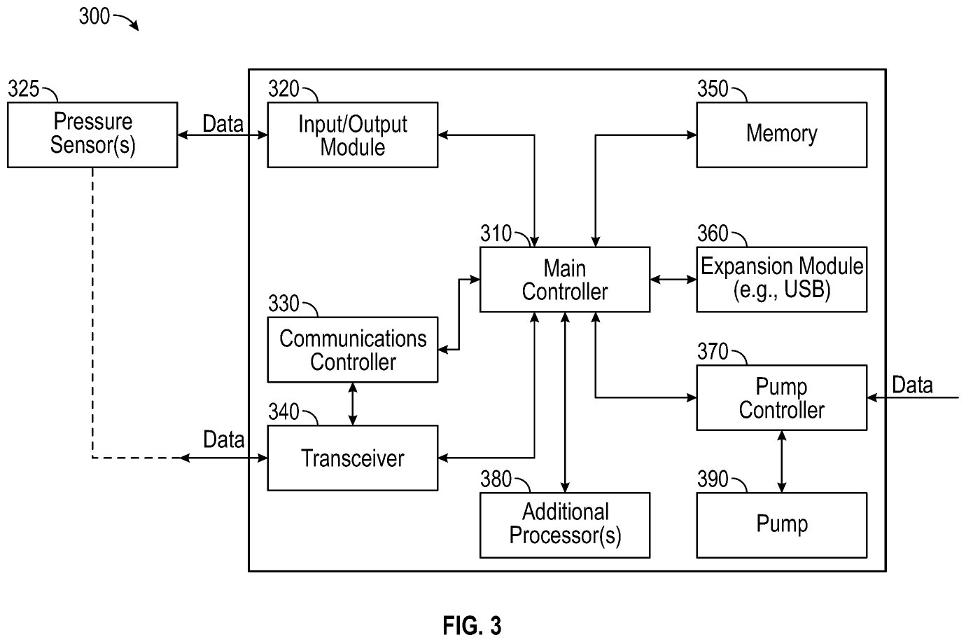

FIG. 3 illustrates a schematic of a control system 300 that can be employed in any of the wound therapy devices described herein, such as in the wound therapy device 110. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controllers 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110. The wound therapy device 110 can be in fluidic communication with one or more wound dressings 406a, 406b (collectively referred to as 406) so as to supply negative pressure to one or more wounds, such as the wounds 104a and 104b. A first fluid flow path can include components providing fluidic connection from the wound therapy device 110 to the first wound dressing 406a. As a non-limiting example, the first fluid flow path can include the path from the wound dressing 406a to the wound therapy device 110 or the path from the first wound dressing 406a to an inlet 446 of a branching attachment (or connector) 444 in fluidic connection with the wound therapy device 110. Similarly, a second fluid flow path can include components providing fluidic connection from the wound therapy device 110 to the second wound dressing 406b.

The system 400 can be similar to the system 100 with the exception that multiple wounds 104a and 140b are being treated by the system 400. The system 400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110 can be fluidically coupled via the tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the dressings 406a, 406b. Once all conduits and dressing components are coupled and operably positioned, the wound therapy device 110 can be activated, thereby supplying negative pressure via the fluid flow paths to the wounds 104a, 104b. Application of negative pressure can be applied until a desired level of healing of the wounds 104a, 104b is achieved. Although two wounds and wound dressing are illustrated in FIG. 4, some implementations of the wound therapy device 110 can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850 or International Publication No. WO2018/167199, each of which is incorporated by reference in its entirety.

### Protection and Isolation of Sensors

Any of the negative pressure wound therapy systems disclosed herein, such as the system 100, can be configured to detect status of the canister. Canister status detection system can function as a fluid detection system to detect the volume of fluid (such as, exudate) within the canister (or fill level of the canister) or whether the canister has reached a full or almost full level of fluid. One or more alarms or alerts can be generated responsive to the detection. A canister full or nearly full alert can be important for a negative pressure therapy system because it can allow a healthcare professional or a user to replace their canister and continue therapy with the least amount of interruptions in the provision of therapy.

Canister fluid detection systems may rely on comparing peak-to-peak voltage measurements obtained from a pressure sensor to threshold values over a certain period to then trigger the canister full or nearly full alert. In some cases, this approach can be unreliable and can have a low tolerance for variations in conditions. For instance, nuisance alarms may be generated when the canister is empty but there are restrictions in flow from the filter assembly.

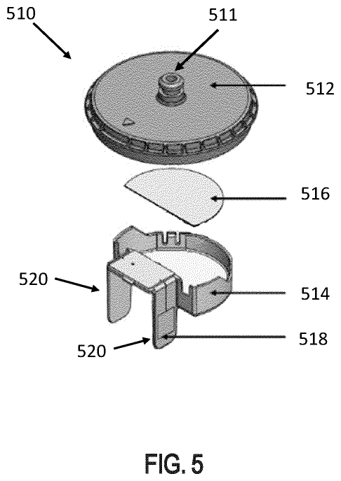

Accordingly, it can be useful to have a more accurate detection method for detecting a full canister condition or a nearly full canister condition. A canister detection system configured for fluid detection can use a device (such as, a fluid level sensor) that communicates with fluid within the canister to detect when the fluid reaches one or more threshold levels within the canister. The canister can incorporate a fluid level sensor within a surface of the canister. For example, the canister can incorporate the fluid level sensor within a cap portion of the canister system. FIG. 5 illustrates a canister cap 510 that can be positioned on a surface of the canister configured to mate or be in a mating arrangement with the negative pressure wound therapy device, such as the pump assembly 160. The canister cap 510 can be positioned to provide fluid communication between the negative pressure source and the interior of the canister. For instance, the canister cap 510 can be positioned at the top of the canister 162, as is shown in FIG. 2A. The pump assembly 160 can be removably attached to the canister cap 510. The canister cap 510 can include an inlet 511 through which fluid aspirated from the wound can enter the interior of the canister.

The canister cap 510 can include a housing formed from a cap top 512 and a cap bottom 514. The canister cap 510 can include a filter 516 positioned between the cap top 512 and the cap bottom 514. A fluid level sensor 518 can be included within the canister cap 510 to communicate with the interior of the canister. The fluid level sensor 518 can include two arms 520 that extend from the cap bottom 514 into the interior of the canister. The arms 520 can be made of conductive material (such as, conductive metal). The arms 520 can be used to interact with the fluid within the canister and create a completed circuit when the arms of the fluid level sensor 518 are in communication with fluid in the canister, which can thereby detect a canister full condition. The fluid level sensor can detect a completed electrical circuit when fluid within the interior of the canister is in contact with the arms of the fluid level sensor. For example, the fluid level sensor can detect fluid collection capacity within the canister when the circuit is open and a canister full condition when the circuit is closed. In other examples, the fluid level sensor can detect fluid collection capacity (or fluid level) within the canister when the circuit is closed and a canister full condition when the circuit is open.

While the fluid level sensor 518 is shown with two downwardly extending arms, the fluid level sensor can include only one arm or any number of arms that extend into the interior of the canister to detect fluid within the canister. In some cases, the fluid level sensor can have any number or extensions or arms as long as at least two separate tracks of conductive material (or electrodes) are present to form the completed electrical circuit.

A reader within the negative pressure wound therapy device can communicate with the fluid level sensor and, responsive to the canister full condition being detected (or responsive to detecting the canister fill level), can provide an indication of a condition of the canister. The reader can also cause a change in the provision of negative pressure wound therapy (such as, cease application of negative pressure) or cause an alert (such as, a canister full alert) in response to the canister full condition being detected by the fluid level sensor. The reader can be positioned within a housing of the negative pressure wound therapy device, such as the pump assembly 160.

The fluid level sensor 518 can include or be part of a detection system configured to communicate wired or wirelessly (such as, using NFC, RFID, etc.). The detection system can utilize fluid level sensor that incorporates a communication device to communicate information from the canister to the device or another remote computing device 334 (such as, the remote computing device 334). The detection system can utilize a communication device for communicating the information from the canister to the device using NFC. NFC is a set of short-range wireless technologies, typically requiring a separation of 10 cm or less (in some cases, 4 cm or less). NFC can involve an initiator (or active tag) and a target (or passive tag). The initiator can actively generate a radio frequency (RF) field that can power the passive tag. In some cases, NFC communication can utilize an NFC reader communicating with a passive NFC tag. The NFC reader can retrieve information stored in the passive NFC tag. The pump assembly 160 can include an NFC reader (which can be located at or near the bottom of the pump assembly 160 housing) and the detection system of the canister (such as, the fluid level sensor 518) can include a passive NFC tag. When the conductive portions of the NFC tag are in contact with fluid within the canister and the circuit is closed, information (such as, a flag) can be stored in memory of the NFC tag. The NFC reader can read such information by communicating with the NFC tag. The NFC reader and NFC tag can include one or more antennas to facilitate wireless communication (for example, facilitate transmission and reception of data). In some cases, the range of communication between the NFC reader and the NFC tag can be about 20 mm (or less or more), which can exceed the distance between the NFC reader and the NFC tag.

Additional details of determining canister status are disclosed in International Patent Application No. PCT/EP2022/060464 (Atty. Docket SMNPH.654WO) titled “COMMUNICATION SYSTEMS AND METHODS FOR NEGATIVE PRESSURE WOUND THERAPY DEVICES,” filed on Apr. 20, 2022; International Patent Application No. PCT/EP2022/060463 (Atty. Docket SMNPH.672WO) titled “CANISTER STATUS DETERMINATION FOR NEGATIVE PRESSURE WOUND THERAPY DEVICES,” filed on Apr. 20, 2022; International Patent Application No. PCT/EP2023/060165 (Atty. Docket SMNPH.712WO) titled “CANISTER STATUS DETERMINATION FOR NEGATIVE PRESSURE WOUND THERAPY DEVICES”, filed on Apr. 19, 2023; U.K. Patent Application No. 2214047.9 (Atty. Docket SMNPH.716GB) titled “CANISTER STATUS DETERMINATION FOR NEGATIVE PRESSURE WOUND THERAPY DEVICES,” filed on Sep. 27, 2022; and U.K. Patent Application No. 2214052.9 (Atty. Docket SMNPH.715GB) titled “CANISTER STATUS DETERMINATION FOR NEGATIVE PRESSURE WOUND THERAPY DEVICES,” filed on Sep. 27, 2022, the disclosure of each of which is hereby incorporated by reference in its entirety.

In some cases, the system (such as, the pump assembly 160) can include memory that stores additional information related to the status of the canister. For example, the additional information may include information about the canister size (such as, 300 mL or 800 mL). In some cases, the additional information can include an indication whether the same canister had been previously attached to the negative pressure wound therapy device. For example, a unique canister identifier can indicate if the same canister is being removed and put back on the negative pressure system. The unique canister identifier can allow for tracking of the use of the canister. In some cases, the additional information stored in the sensor device in the canister can include tracking information entered into the device, such as the time and/or date when the canister was connected to the pump assembly, when the canister was removed from the pump assembly, and/or the number of hours of operating time. Additional details of the status of the canister are disclosed in International Patent Application No. PCT/EP2022/060459 (Atty. Docket SMNPH.683WO), titled “INTELIGENT DISPOSABLE DEVICES FOR WOUND THERAPY AND TREATMENT,” filed on Apr. 20, 2022, and incorporated by reference in its entirety.

Because the fluid level sensor 518 is positioned in the canister, it can be exposed to liquid, humidity, or the like. Such exposure to a conductive substance (for instance, exudate) can cause one or more of electrostatic discharge (ESD), electrical fast transients (EFT), high differential voltages, high currents, or the like. These may cause malfunction or degradation of the fluid level sensor 518 or can cause leakage current to flow through the patient, which can be dangerous to the patient. For instance, leakage current may flow as a result of the patient touching a source of electric potential (such as, an exposed wire or mains) and due to a difference in the electric potential between such source (and the patient's electric potential due to the patient touching the source) and the fluid level sensor 518 (which, when activated and powered on, can operate as at a certain electric potential, such as 5V, different from the patient's electric potential). Flow of the leakage current can be dangerous to the patient (for instance, if conducted through the chest and defibrillating the heart). Thus, it can be advantageous to design the fluid level sensor 518 with mechanisms to protect the electronics and the patient from such negative effects.

The approaches described herein can isolate the patient and prevent any leakage current from conducting through the patient's body. Advantageously, this can facilitate compliance with the applicable regulations, such as the IEC 60601-1 safety standard that specifies that the maximum patient leakage current in certain cases cannot exceed 50 microamperes.

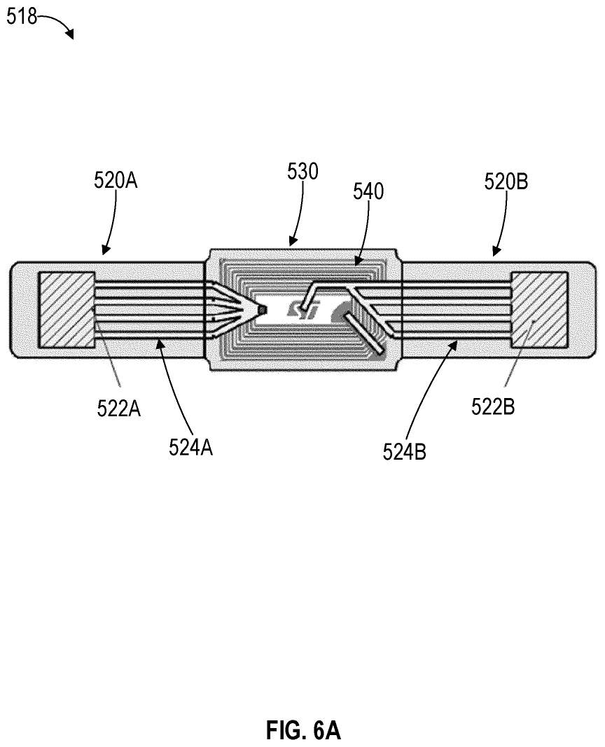

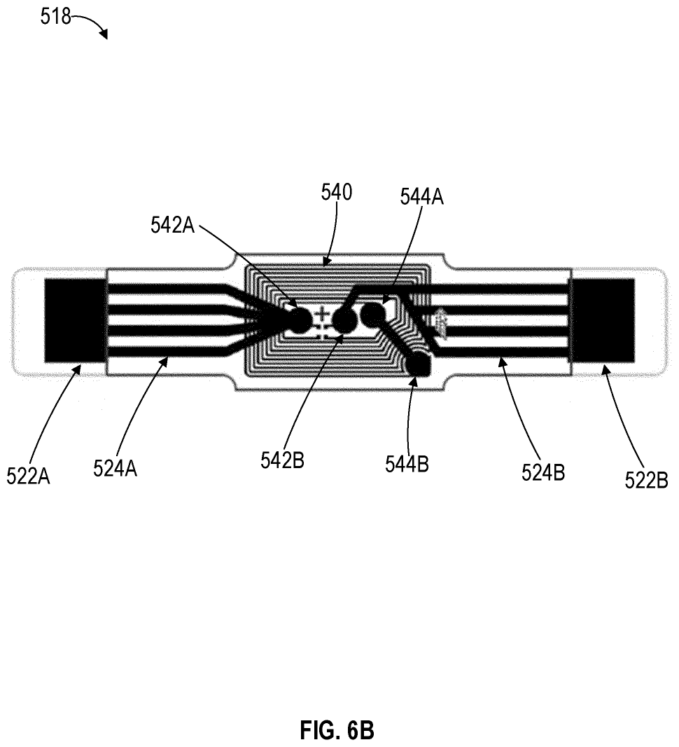

FIGS. 6A and 6B illustrate electronic components of the fluid level sensor 518 with arms 520A and 520B. Each arm can include sensing pads (or sensing electrodes) 522A and 522B made out of electrically conductive material(s). The sensing pads 522A and 522B can be electrically exposed so as to come into contact with the fluid in the canister. Electronic circuitry 530 can be configured to process signals detected by the sensing pads 522A and 522B and communicate with an external device, such as the reader. For example, detecting high impedance (or open circuit) across the sensing pads 522A and 522B can indicate that the fluid level has not reached the sensing pads. As another example, detecting low impedance (or short circuit) across the sensing pads 522A and 522B can indicate that the fluid level has reached the sensing pads, which can be indicative of a fluid level within the canister or that the canister is full (or nearly full). Connections 524A and 524B can connect the sensing pads 522A and 522B to the electronic circuitry 530. The electronic circuitry 530 can be positioned between the arms 520A and 520B, as illustrated in FIGS. 6A and 6B.

Connections 524A and 524B can be electrically connected to the electronic circuitry 530, which can detect whether there is an open circuit or short circuit across the connections. Connections 524A and 524B can be made of electrically conductive material(s). Open circuit can be indicative of the fluid in the canister not reaching a particular level and short circuit can be indicative of the fluid in the canister reaching the particular level. Electronic circuitry 530 can include logic circuitry configured to process signals detected by the sensing pads 522A and 522B. Logic circuitry can be packaged as an integrated circuit (IC). Electronic circuitry 530 can include one or more external connectors 542A and 542B that are connected to the connections 524A and 524B. For instance, when the logic circuitry is provided in an IC package (sometimes referred to herein as “IC”), connectors 542A and 542B can be pins, pads, or the like of the IC package.

Connections 524A and 524B and electronic circuitry 530 can be electrically isolated. To isolate the connections 524A and 524B from liquid, humidity, or the like, the connections can be protected by non-conductive material(s), such as coated with non-conductive material(s) or encapsulated in non-conductive material(s). To isolate the electronic circuitry 530 from the liquid, humidity, or the like, the electronic circuitry (such as, the IC) can be protected by non-conductive material(s), such as coated with non-conductive material(s) or encapsulated in non-conductive material(s). Non-conductive material described herein can include potting, glue, resin, paint, coating, mask, or the like.

The electronic circuitry 530 can include communication circuitry configured to transmit canister status information, for instance, wirelessly to the pump assembly 160. The communication circuitry can utilize an antenna 540 (illustrated as a loop antenna). The antenna 540 can be an antenna positioned on a printed circuit board (PCB). For instance, the antenna 540 can be configured for NFC communications. Logic circuitry and communication circuitry can be packaged in a single IC package. The antenna 540 can be connected to the communication circuitry of the IC using connectors 544A and 544B, which can be pins, pads, or the like of the IC package. In some implementations, the antenna 540 can be connected to the communication circuitry using a single connector, and in such cases any references herein to connectors 544A and 544B should be understood to refer to the single connector.

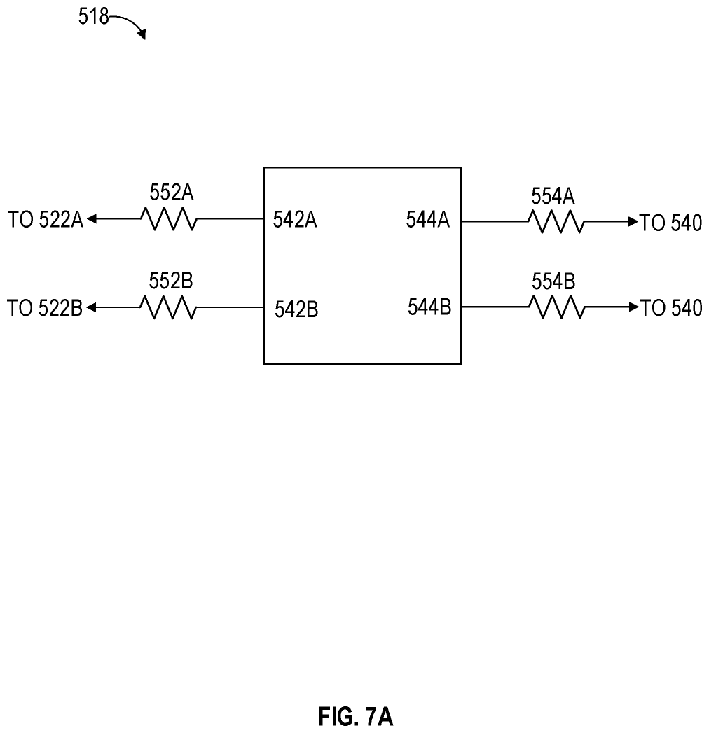

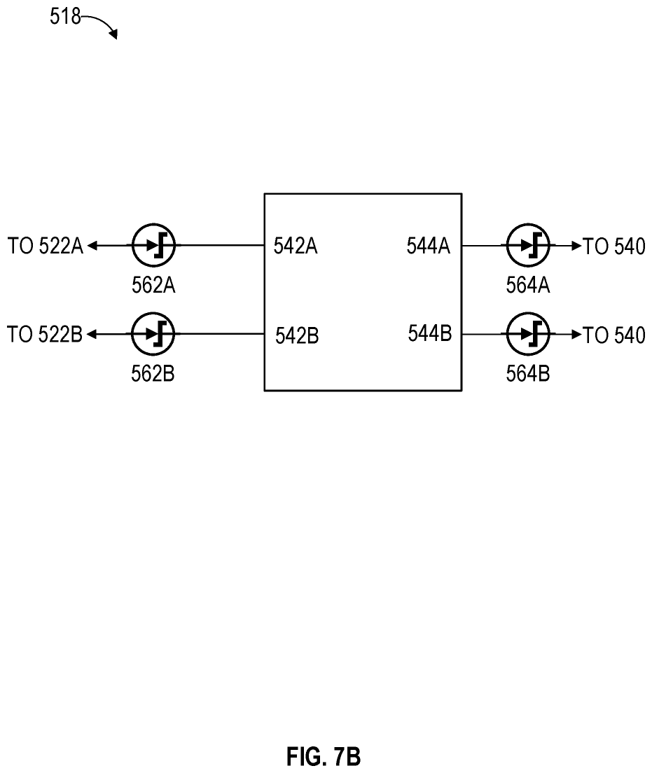

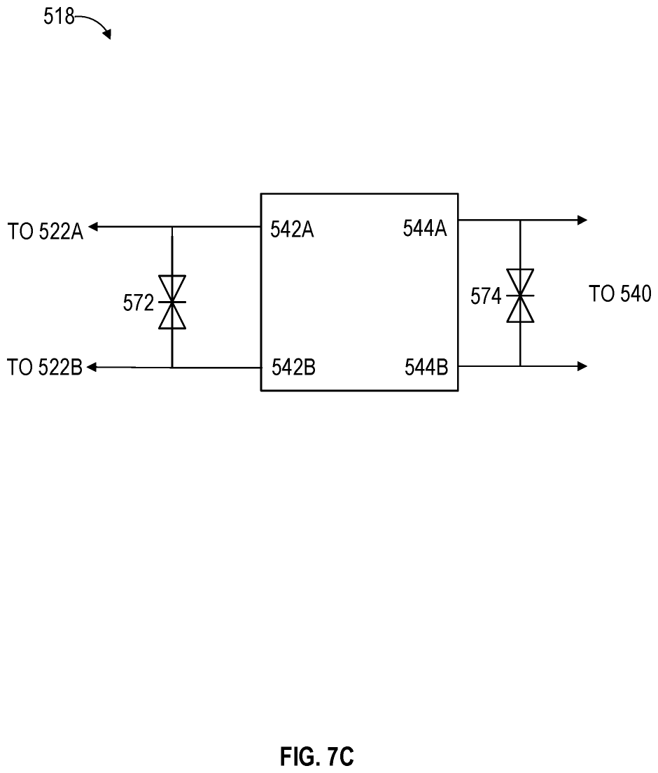

Exposure to liquid or humidity can undesirably cause leakage current in the electronic circuitry 530. As described herein, leakage current can flow from one or both pins 542A and 542B to a remote node having different voltage potential or to a ground (for instance, via the exudate). Leakage current can be dangerous to the patient (for example, can cause discomfort, injury, or even death). This may occur when power is provided to the electronic circuitry 530, for instance, as a result of power transfer via the NFC protocol (as the fluid level sensor 518 can be configured as a passive NFC tag that does not include a power source), which can create a difference in the electric potential that can facilitate flow of leakage current. As described herein, such difference in the electrical potential can be created due to the patient touching an exposed wire or mains and the electronic circuitry 530 operating at a different potential. To minimize or eliminate leakage current, one or more resistors can be added to one or both of the pins 542A and 542B. This can reduce any leakage current since any such current would be conducted through the one or more resistors and, as a result, reduced. The one or more resistors can be added in series with one or both pins 542A and 542B. FIG. 7A illustrates resistors 552A and 552B added in series to the pins 542A and 542B. Resistors 552A and 552B can have different or same impedance values.

In some cases, a threshold impedance for detecting a short circuit should correspond to the impedance of the fluid (such as, wound exudate) expected to fill the canister. Testing of stimulated exudate (0.9% saline) has revealed impedance of about 66Ω. The threshold impedance can be set to a value that is less than this impedance. Since impedance of simulated exudate is a small, the threshold would be set to a relatively small impedance (for example, about 50Ω). Because of this, in some cases, impedance values of the resistors 552A and 552B would need to be small and much lower than the threshold impedance (otherwise, accuracy of detecting a short circuit and fluid level would degrade). On the other hand, to provide effective leakage current protection, impedance values of the resistors 552A and 552B may need to be as large as possible in order to reduce any leakage current. In some cases, impedance values of the resistors 552A and 552B can be set to 15-30% of the threshold impedance. For instance, assuming threshold impedance of about 5052, impedance values of the resistors 552A and 552B can be set to about 10Ω. In some implementations, combined impedance value of the resistors 552A and 552B can be set to 15-30% of the threshold impedance.

With reference to FIG. 7A, pins 544A and 544B that provide connection to the antenna 540 can be similarly protected with one or more resistors 554A and 554B. These resistors can have same or different impedance values, which may be the same as impedance values of the resistors 552A and 552B or be different.

Additionally or alternatively, fluid level sensor 518 electronics can be designed so that the electrodes 522A and 522B or the electronic circuitry 530 do not come into direct contact with the wound exudate (and the patient's body). Instead, magnetic coupling using one or more mutually coupled inductors or transformers can be used to sever any direct electrical connection between the electrodes or the electronic circuitry and the patient.

As described herein, it can be advantageous to protect the electronic circuitry 530 from electromagnetic discharge (ESD), including, for example, electrical fast transients (EFT), or prevent flow of any leakage current. This may be particularly important in cases the IC does not include a ground connection. With reference to FIG. 7B, this can be accomplished by, additionally or alternatively, adding one or more diodes 562A and 562B to the pins 542A and 542B. The diodes 562A and 562B can limit (or clamp) the voltage provided to the electronic circuitry 530, for example, to approximately 3V, 5V, or another suitable voltage for which the electronic circuitry is configured. As a result, the magnitude of any ESD pulses can be regulated.

The diodes 562A and 562B can include one or more zener diodes. In some cases, the diodes 562A and 562B can be transient voltage suppressors (TVS) diodes, which can include zener diode(s). The diodes 562A and 562B can be selected to provide sufficient protection to comply with applicable regulations, such to withstand ±15kV air discharge as provided by IEC 60601-1 safety standard.

FIG. 7B illustrate unidirectional diodes 562A and 562B. Diodes 562A and 562B can be similar or different. With reference to FIG. 7B, pins 544A and 544B that provide connection to the antenna 540 can be similarly protected with one or more diodes 564A and 564B. These diodes can be same as the diodes 562A and 562B or be different.

FIG. 7C illustrates a bidirectional diode 572 positioned to protect the pins 542A and 542B from ESD. The bidirectional diode 572 can be positioned across the pins 542A and 542B. As described herein, the bidirectional diode 572 can be a TVS diode. Bidirectional diode 574 can be used to similarly protect the pins 544A and 544B. Bidirectional diodes 572 and 574 can be the same or different.

Additionally or alternatively, the electronic circuitry 530 of the fluid level sensor 518 can be powered on periodically for a short period of time (such as, in short bursts). Electrical current does not propagate instantly through the human body, but rather it can take up to fractions of a second for the cells of the body to form pathways (such as, ion channels) for conducting the electrical current. As long as the fluid level sensor 518 is powered on for a shorter duration of time than a threshold duration of time associated with the minimum duration of time that the body can be safely exposed to a leakage current of a maximum possible expected magnitude (for example, 15-20 amperes of mains current), patient safety would not be compromised. For instance, in some cases serious injury or death are unlikely to occur provided that the leakage current does not exceed 1 ampere for 30 milliseconds, 15 amperes for 2 milliseconds, or 20 amperes for 1.5 milliseconds.

As an example, let's assume that the electronic circuitry 530 is an NFC tag, such as a passive NFC tag. The electronic circuitry 530 can wirelessly receive commands from an external device (such as, the NFC reader as described herein). The commands can activate and provide power to the electronic circuitry 530, read the detected state (such as, open circuit or closed circuit across the sensing pads 522A and 522B), and deactivate and power off the electronic circuitry 530. In some cases, such sequence of powering on, reading, and powering off may take less than about 1.5 milliseconds, which can be shorter than the threshold duration of time. This way, instead of powering on the electronic circuitry 530 for long period of time, the electronic circuitry can be powered on periodically in short bursts to safely determine the level of fluid in the canister.

In case the electronic circuitry 530 becomes unresponsive after being powered on (and as a result could not be powered off with a command), a bleeder resistor can be positioned in the electronic circuitry 530 to dissipate any stored electric charge that had been applied to the electronic circuitry. The bleeder resistor can be connected across an energy storing electrical component (such as, a capacitor) of the electronic circuitry 530 in order to discharge such energy storing electrical component.

The above-described approach for activation and communication in short bursts can provide protection against malfunction of the electronic circuitry 530 due to, for example, ingress of exudate into the electronic circuitry 530. Additionally, the above-described approach for activation and communication in short bursts can protect the patient against any leakage current caused by the circuitry coming into contact with the exudate.

In some implementations, the electronic circuitry 530 can be positioned between the arms 520A and 520B. Rigidity of at least some components of the electronic circuitry 530 can be increased to protect such one or more components from bending, for example, over the canister cap 510. For example, base material can be used to increase the rigidity. In some cases, base material for printed circuit board (PCB) technology (such as, fiberglass with epoxy (FR4)) can be added to increase the rigidity. The IC can be placed on a PCB of the electronic circuitry 530 (for instance, using conductive glue), and base material can be added (for instance, below the IC) to increase the rigidness of the PCB (and the IC). This may be particularly pertinent to implementations in which the PCB of the electronic circuitry 530 is flexible or substantially flexible. As a result, any conductivity failures can be eliminated or limited.

Additionally or alternatively to the electric approaches for protection against leakage current, such as those described in reference to FIGS. 6A, 6B, and 7A to 7C, mechanical separation of the fluid level sensor 518 electronics from the patient can be utilized. The canister can include an aperture structure that can isolate the fluid level sensor 518 electronics. The aperture structure can include a weir, which can be a liquid flow control structure that allows liquid to flow over its top and provides a drop. The aperture structure can be positioned in or proximal to the canister inlet, such as the inlet 511 illustrated in FIG. 5. For example, the aperture structure can be positioned within the canister and proximal to the canister inlet or outside the canister (such as, in a conduit) and proximal to the canister inlet. As the fluid enters the canister through the inlet, the fluid drops under gravity from the aperture structure that ensures separation of the liquid stream into drops, rather than permitting a continuous liquid stream. This can generate an air gap separation between the fluid flow path connected to the patient and the bulk of the liquid stored in the interior volume of the canister (which comes into contact with the fluid level sensor 518 electronics).

The aperture structure can be made out of a hydrophobic material so that the structure would not degrade when coming into contact with liquid. In some instance, the aperture structure can be made out of polytetrafluoroethylene (PTFE).

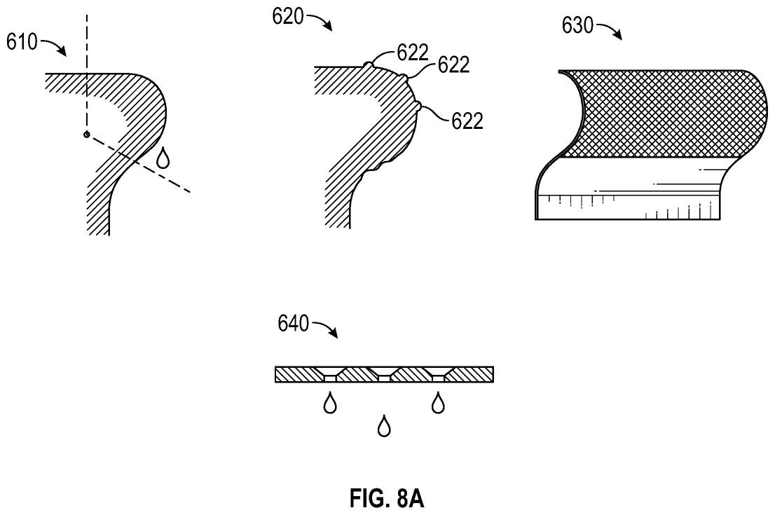

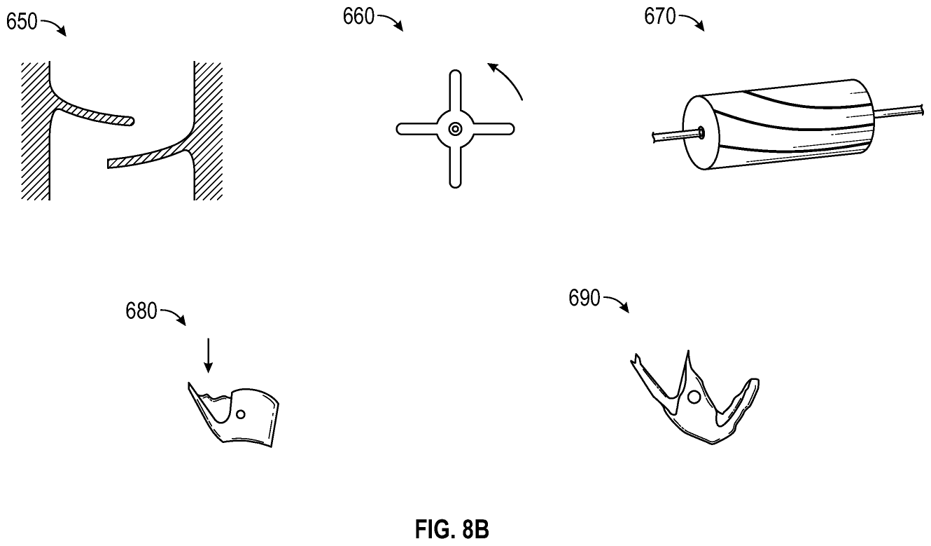

In FIGS. 8A to 8B, fluid aspirated from the wound would be positioned above any of the illustrated aperture structures and the interior volume of the canister in which the fluid level sensor 518 is placed would be positioned below. With reference to FIG. 8A, an aperture structure 610 can include a curved edge. The curved edge can continue across orientations extending at a minimum orthogonal distance to the primary orientation of the wound therapy device (such as, the upright orientation). Liquid aspirated from the wound would be separated into drops, as is illustrated. An aperture structure 620 can be similar to the aperture structure 610, but the edge can be knurled and include one or more bumps or ridges 622. The ridges 622 can act as weirs. The ridges 622 can have different orientations. An aperture structure 630 can be a knurled breaker. The surface of the aperture structure 630 can be include ridges that separate aspirated liquid into drops. An aperture structure 640 can be a sifter or a grille with one or more apertures that permit liquid drops to pass through.

With reference to FIG. 8B, an aperture structure 650 can include a plurality of extensions or arms that would separate liquid aspirated from the wound into drops. An aperture structure 660 can be a rotating wheel that would break the flow of liquid aspirated from the wound. If the rate of fluid flow is not sufficient to rotate the wheel, such flow would be generate a solid stream in the first place. An aperture structure 670 illustrates an alternate design of the wheel. The illustrated wheel can be knurled. An aperture structure 680 can be a pivoted bucket that fills and subsequent tips over (or rocks over) to release the fluid into the interior volume of the canister. An aperture structure 690 includes two pivoted buckets one of which (for instance, the left one) can be configured to be filled and subsequently tip over to fill the other pivoted bucket (for instance, the right one). This way, there can be multiple fill volumes so that, on tipping, the liquid drops into a secondary bucket that mirrors the primary bucket and alternates between the two bucket volumes as the filling continues. Advantageously, the described mechanical approaches can eliminate any residual leakage current flowing through the patient as result of conductivity to a remote earth ground or potential.

The following approaches can be utilized additionally or alternatively to the above approaches for protection and isolation. The negative pressure wound therapy system may be configured to provide therapy when one or more of the pump assembly 160 or canister are in proper orientation, such as vertically positioned. Orientation of one or more of the pump assembly 160 or canister can be monitored. For example, one or more accelerometers or gyroscopes can be used to monitor the orientation of one or more of the pump assembly 160 or canister. In response to detecting that one or more of the pump assembly 160 or canister are not in correct orientation during provision of therapy, the fluid level sensor 518 (which may be configured as a passive NFC tag without a power source) may not be powered on. For example, electronic circuitry (such as, the control system 300) of the pump assembly may monitor the orientation and not provide power to the fluid level sensor 518. This can eliminate or limit risk of flow of any leakage current.

Additional details of determining device orientation are disclosed in International Patent Application No. PCT/EP2022/079091, filed on Oct. 19, 2022 and titled “USER-FRIENDLY NEGATIVE PRESSURE WOUND THERAPY DEVICES AND METHODS OF OPERATING SUCH DEVICES” (Atty. Docket SMNPH.677WO), the disclosure of which is incorporated by reference in its entirety.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound monitoring and/or treatment device in which, during operation, electronic components may come into contact with conductive fluid or other conductive substances. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. As another example, systems, devices, and/or methods disclosed herein can be used with a wound debridement system, patient monitoring system, or the like. As yet another example, systems, devices, and/or methods disclosed herein can be used with any system that contacts and analyzes human exudate. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A negative pressure wound therapy system comprising:
a negative pressure wound therapy device including a negative pressure source configured to aspirate, via a fluid flow path, fluid from a wound of a patient covered by a wound dressing;
a canister positioned in the fluid flow path and configured to store at least some of the fluid aspirated by the negative pressure source; and
a fluid level sensing circuitry positioned at least partially in the canister, the fluid level sensing circuitry comprising:
first and second electrodes configured to come into contact with fluid stored in the canister when the fluid reaches the first and second electrodes;
a processing circuitry comprising first and second connectors connected to the first and second electrodes, the processing circuitry configured to determine a level of fluid stored in the canister based on a signal received from the first and second electrodes, the processing circuitry further configured to be powered on periodically for a duration of time that is sufficient to determine the level of fluid stored in the canister and does not exceed a threshold duration of time associated with a minimum duration of time that the patient can be safely exposed to a leakage current; and
first and second resistors connected to the first and second connectors and configured to protect the patient against the leakage current.

2. The negative pressure wound therapy system of claim 1, wherein:
an impedance across the first and second electrodes decreases responsive to the fluid in the canister reaching the first and second electrodes;
the processing circuitry is configured to determine the level of fluid responsive to detecting that the impedance across the first and second electrodes is below an impedance threshold indicative of a short circuit across the first and second electrodes, the short circuit formed by the fluid in the canister reaching the first and second electrodes; and
impedance of the first and second resistors is lower than the impedance threshold.

3. The negative pressure wound therapy system of claim 2, wherein the impedance of the first and second resistors comprises 15-30% of the impedance threshold.

4. The negative pressure wound therapy system of claim 1, wherein the processing circuitry comprises an integrated circuit (IC), and wherein the first and second connectors comprise first and second pins of the IC.

5. The negative pressure wound therapy system of claim 1, wherein:
the fluid level sensing circuitry comprises an antenna configured to wirelessly receive power from the negative pressure wound therapy device and communicate the level of fluid stored in the canister to the negative pressure wound therapy device;
the processing circuitry comprises third and fourth connectors connected to the antenna; and
the fluid level sensing circuitry comprises third and fourth resistors connected to the third and fourth connectors and wherein the third and fourth resistors are configured to protect against leakage current.

6. The negative pressure wound therapy system of claim 5, wherein the processing circuitry comprises an integrated circuit (IC), wherein the first and second connectors comprise first and second pins of the IC, and wherein the third and fourth connectors comprise third and fourth pins of the IC.

7. The negative pressure wound therapy system of claim 5, wherein the fluid level sensing circuitry further comprises at least one first diode connected to the first and second connectors, and wherein the at least one first diode is configured to protect the fluid level sensing circuitry against electrostatic discharge (ESD).

8. The negative pressure wound therapy system of claim 7, wherein the at least one first diode comprises a first bidirectional transient voltage suppressor (TVS) diode connected across the first and second connectors.

9. The negative pressure wound therapy system of claim 7, wherein the fluid level sensing circuitry further comprises at least one second diode connected to the third and fourth connectors, and wherein the at least one second diode is configured to protect the fluid level sensing circuitry against electrostatic discharge (ESD).

10. The negative pressure wound therapy system of claim 9, wherein the at least one first diode comprises a first bidirectional transient voltage suppressor (TVS) diode connected across the first and second connectors, and wherein the at least one second diode comprises a second bidirectional transient voltage suppressor (TVS) diode connected across the third and fourth connectors.

11. (canceled)

12. The negative pressure wound therapy system of claim 1, wherein:
the negative pressure wound therapy device comprises electronic circuitry configured to monitor orientation of at least one of the negative pressure wound therapy device or the canister;
the fluid level sensing circuitry is further configured to receive power wirelessly from the electronic circuitry of the negative pressure wound therapy device; and
the electronic circuitry of the negative pressure wound therapy device is further configured to not provide power to the fluid level sensing circuitry responsive to a detection that at least one of the negative pressure wound therapy device or the canister is in an incorrect orientation for aspirating fluid from the wound.

13. The negative pressure wound therapy system of claim 12, wherein the incorrect orientation comprises orientation other than vertical orientation.

14. The negative pressure wound therapy system of claim 12, wherein the fluid level sensing circuitry is configured to operate as a passive near field communication (NFC) device.

15. (canceled)

16. The negative pressure wound therapy system of claim 1, wherein the canister comprises a cap configured to be attached to a housing of the negative pressure wound therapy device, and wherein the fluid level sensing circuitry is positioned in the cap, and wherein the first and second electrodes are oriented downward toward an interior volume of the canister.

17. (canceled)

18. (canceled)

19. The negative pressure wound therapy system of claim 16, wherein the processing circuitry comprises an integrated circuit (IC) reinforced with a base material that comprises fiberglass with epoxy (FR4).

20. (canceled)

21. A negative pressure wound therapy system comprising:
a negative pressure wound therapy device including a negative pressure source configured to aspirate, via a fluid flow path, fluid from a wound of a patient covered by a wound dressing;
a canister positioned in the fluid flow path and configured to store at least some of the fluid aspirated by the negative pressure source; and
a fluid level sensing circuitry positioned at least partially in the canister, the fluid level sensing circuitry comprising:
first and second electrodes configured to come into contact with fluid stored in the canister when the fluid reaches the first and second electrodes; and
a processing circuitry configured to determine a level of fluid stored in the canister based on a signal received from the first and second electrodes, the processing circuitry further_configured to be powered on periodically_for a duration of time that is sufficient to determine the level of fluid stored in the canister and does not exceed a threshold duration of time associated with a minimum duration of time that the patient can be safely exposed to a leakage current.

22. The negative pressure wound therapy system of claim 21, wherein the negative pressure wound therapy device is configured to power on the processing circuitry and receive the level of fluid stored in the canister.

23. The negative pressure wound therapy system of claim 21, wherein the fluid level sensing circuitry further comprises a resistor configured to dissipate power stored by the processing circuitry.

24. A negative pressure wound therapy system comprising:
a negative pressure wound therapy device including a negative pressure source configured to aspirate, via a fluid flow path, fluid from a wound of a patient covered by a wound dressing;
a canister positioned in the fluid flow path and configured to store at least some of the fluid aspirated by the negative pressure source, the canister comprising an inlet configured to be connected to the negative pressure wound therapy device;
a fluid level sensing circuitry positioned at least partially in the canister, the fluid level sensing circuitry comprising:
first and second electrodes configured to come into contact with fluid stored in the canister when the fluid reaches the first and second electrodes; and
a processing circuitry configured to determine a level of fluid stored in the canister based on a signal received from the first and second electrodes; and
an aperture structure positioned in the inlet and configured to separate a stream of fluid aspirated by the negative pressure source into drops to attain an air gap separation between the stream of the fluid and the fluid stored in the canister.

25. The negative pressure wound therapy system of claim 24, wherein the aperture structure comprises at least one of a curved edge, a curved edge with one or more ridges, a surface with one or more ridges, a surface one or more openings, a plurality of arms, a rotating wheel, or at least one pivoted bucket.

26. (canceled)
